(19) United States
(12) Patent Application Publication
(10) Pub. No.: US 2018/0280430 A1
(43) Pub. Date: Oct. 4, 2018

(54) COMPOSITION FOR IMPROVED PERFORMANCE

(71) Applicant: Balchem Corporation, Layton, UT (US)

(72) Inventor: Jonathan David Bortz, Layton, UT (US)

(21) Appl. No.: 15/765,403

(22) PCT Filed: Sep. 29, 2016
(86) PCT No.: PCT/US16/54492

§ 371 Date: Apr. 2, 2018

Related U.S. Application Data
(60) Provisional application No. 62/236,292, filed on Oct. 2, 2015.

(51) Int. Cl. A61K 33/30; A61K 33/06; A61K 33/26; A61K 31/198; A61K 31/205; A61K 35/744; A61K 35/745; A61K 35/747; A61K 31/14; A23L 33/00; A23L 33/175; A23L 33/185; A23L 33/105; A23L 33/16; A23L 33/135; A23L 33/115

(52) U.S. Cl. CPC A61K 33/30; A23V 2002/00; A61K 33/26; A61K 31/198; A61K 31/205; A61K 35/744; A61K 35/745; A61K 35/747; A61K 31/14; A23L 33/40; A23L 33/175; A23L 33/185; A23L 33/105; A23L 33/16; A23L 33/135; A23L 33/115; A61K 33/06

(57) ABSTRACT

The present invention encompasses single dosage form compositions comprising component (a) and component (b). Component (a) is a combination of proteins and carbohydrates in a weight ratio that is at least about 0.5:1. Component (b) is magnesium, zinc, and/or choline. The invention also encompasses use of the single dosage form compositions to improve exercise-related performance.

### CROSS REFERENCE TO RELATED APPLICATIONS

This application claims the priority of PCT Application PCT/US2016/054492, filed Sep. 29, 2016, which claims the benefit of U.S. Provisional Application 62/236,292, filed Oct. 2, 2015, each of which is hereby incorporated by reference in its entirety.

### FIELD OF THE INVENTION

The present invention encompasses single dosage form compositions comprising component (a) and component (b). Component (a) is a combination of proteins and carbohydrates in a weight ratio that is at least about 0.5:1. Component (b) is magnesium, zinc, and/or choline. The invention also encompasses use of the single dosage form compositions to improve exercise-related performance.

### BACKGROUND OF THE INVENTION

Incorporating good dietary practices, as part of an exercise program can optimize a subject's performance during exercise. It can be difficult, however, for a person routinely exercising to ingest a sufficient amount of calories and/or the proper balance of carbohydrates, proteins, and other nutrients. Despite the interest in sports nutrition and the commercial market for nutritional supplements, there remains a need in the art for a well-balanced sports nutrition supplement that is in a single dosage form and delivers adequate amounts of labeled ingredients to effect a physiologic benefit.

### DETAILED DESCRIPTION

The present invention provides a single dosage form composition comprising carbohydrates and proteins in discrete ratios and amounts. Compositions of the invention also contain zinc, magnesium, and/or choline. Applicants have discovered the specific combinations of these components, as disclosed herein, are uniquely beneficial to a subject routinely exercising.

### I. Compositions

One aspect of the invention encompasses single dosage form composition comprising component (a) and component (b). Component (a) is combination of proteins and carbohydrates in a weight ratio that is at least about 0.5:1. Component (b) is magnesium, zinc, and/or choline. A single dosage form composition of the invention may further comprise one or more additional components including, but not limited to, creatine; carnitine; iron; calcium; phosphorous; potassium; sodium; other vitamins, minerals, nutrients, and/or salts; amino acids; fats; plant extracts; flavorings; one or more probiotics; and excipients.

Another aspect of the invention encompasses a supplement comprising component (a) and component (b), wherein the amount of component (a) and component (b) present in the supplement produces a beneficial effect on a measure of exercise performance in a subject that has ingested the supplement. Component (a) is a combination of proteins and carbohydrates in a weight ratio that is at least about 0.5:1. Component (b) is magnesium, zinc, and/or choline. A supplement of the invention may further comprise one or more additional components including, but not limited to, creatine; carnitine; iron; calcium; phosphorous; potassium; sodium; other vitamins, minerals, nutrients, and/or salts; amino acids; fats; plant extracts; flavorings; one or more probiotics and excipients.

Another aspect of the invention encompasses a supplement comprising component (a) and component (b), wherein the amount of component (a) and component (b) present in the supplement produces a beneficial effect on one or more gastrointestinal symptom in a subject that has ingested the supplement. Component (a) is a combination of proteins and carbohydrates in a weight ratio that is at least about 0.5:1. Component (b) is magnesium, zinc, and/or choline. A supplement of the invention may further comprise one or more additional components including, but not limited to, creatine; carnitine; iron; calcium; phosphorous; potassium; sodium; other vitamins, minerals, nutrients, and/or salts; amino acids; fats; plant extracts; flavorings; one or more probiotics and excipients.

As used herein, the term “single dosage form composition” refers to the amounts and types of components comprising a single dosage form. The term “dosage form” refers to a formulation of the components in physical form designed to allow the accurate and efficient administration to a subject, preferably to a human. Suitable dosage forms are those that are orally administered. Non-limiting examples of suitable dosage forms include powders, beads, pellets, granules, capsules, tablets, pills, lozenges, soluble films, elixirs, syrups, solutions, suspensions, emulsions, semisolids and gels. Pellets, powders, beads and granules may be contained within a capsule, wet massed and molded into a pill, compressed into a tablet, incorporated into a food product, or packaged for incorporation into a food product or for reconstitution (e.g. as a drink, shake, etc. with any suitable liquid). Capsules may be a one-piece or two-piece capsule, and have a soft or hard shell. Non-limiting examples of tablets include a suspension tablet, a chewable tablet, an effervescent tablet, and an orally disintegrating tablet. Semisolids may include, but are not limited to, gel-filled chews and gelatinous chews. Food products include, but are not limited to, bars, shakes, drinks, and the like.

The term “supplement,” as used herein, refers to a composition to be ingested that contains one or more components intended to add further nutritional value to the diet of a subject (i.e. to “supplement” the diet). A single dosage form composition as provided herein may also be referred to as a supplement because the compositions of the invention are orally administered and contain components that can add further nutritional value to the diet of a subject.

The term “subject” refers to a mammal, preferably a human. In embodiments where a supplement of the invention produces a beneficial effect on a measure of exercise performance in a subject, preferably the subject is a human that regularly exercises (e.g. at least about 30 minutes per day, approximately three times per week). In an exemplary embodiment, the subject is a human that is regularly involved in moderate levels of exercise (e.g. about 1 to about 3 hours per day of exercise in one or more workout, at least four times per week). In another exemplary embodiment, the subject is a human that is regularly involved in high levels of exercise (e.g. 3 or more hours per day of exercise in one or more workout, at least five times per week). It is understood that when the subject is not a human (e.g. a laboratory animal), the duration of exercise can and will change according to methods known in the art.

The amount and types of the components according to this disclosure are described throughout the specification and examples. The amount of any component contained in a single dosage form composition as described herein may be understood to be the amount in a single discrete dosage form such as a tablet, capsule, or lozenge. Alternatively, when the dosage form is in a non-discrete form, (e.g. beads, granules, pellets, powders, elixirs, syrups, solutions or suspensions), the amount of any ingredient contained therein will be understood to be the amount in a specific volume, (e.g., 10 ml, 100 ml, 1000 ml, etc.) or a specific weight (e.g. 30 g, 35 g, 40 g, 45 g, 50 g, 55 g, 60 g, 65 g, 70 g, etc.) providing a single dose. It should be recognized that where a combination of components, including specific amounts of these components, is described with one dosage form that the same combination could be used for any other suitable dosage form. Moreover, it should be understood that one of skill in the art would, with the teachings found within this application, be able to make any of the dosage forms listed above by combining the amounts and types of ingredients administered as a combination in a single dosage form or separate dosage form and administered together as described in the different sections of the specification.

A single dosage form composition of the invention comprises a weight ratio of proteins to carbohydrates that is at least about 0.5:1. For example, the ratio may be about at least about 0.6:1, at least about 0.7:1, at least about 0.8:1, at least about 0.9:1, at least about 1:1, at least about 1.1:1, at least about 1.2:1, at least about 1.3:1, at least about 1.4:1, at least about 1.5:1, at least about 1.6:1, at least about 1.7:1, at least about 1.8:1, at least about 1.9:1, or at least about 2:1.

In some embodiments, a single dosage form composition of the invention comprises a weight ratio of proteins to carbohydrates that is about 0.5:1 to about 2:1. In other embodiments, a single dosage form composition of the invention comprises a weight ratio of proteins to carbohydrates that is about 0.5:1 to about 1.75:1, or about 0.75:1 to about 2:1. In other embodiments, a single dosage form composition of the invention comprises a weight ratio of proteins to carbohydrates that is about 0.5:1 to about 1.5:1, about 0.75:1 to about 1.75:1, or about 1:1 to about 2:1. In other embodiments, a single dosage form composition of the invention comprises a weight ratio of proteins to carbohydrates that is about 0.5:1 to about 1.25:1, about 0.75:1 to about 1.5:1, about 1:1 to about 1.75:1, or about 1.25:1 to about 2:1. In other embodiments, a single dosage form composition of the invention comprises a weight ratio of proteins to carbohydrates that is about 0.5:1 to about 1:1, about 0.75:1 to about 1.25:1, about 1:1 to about 1.5:1, about 1.25:1 to about 1.75:1, or about 1.5:1 to about 2:1.

In certain embodiments, a single dosage form composition of the invention comprises a weight ratio of proteins to carbohydrates that is about 0.5:1, about 0.6:1, about 0.7:1, about 0.8:1, or about 0.9:1. In certain embodiments, a single dosage form composition of the invention comprises a weight ratio of proteins to carbohydrates that is about 1:1, about 1.1:1, about 1.2:1, about 1.3:1, or about 1.4:1. In certain embodiments, a single dosage form composition of the invention comprises a weight ratio of proteins to carbohydrates that is about 1.5:1, about 1.6:1, about 1.7:1, about 1.8:1, or about 1.9:1.

Other aspects of the composition are described in further detail below.

### (a) Carbohydrates

A single dosage form composition of the invention comprises carbohydrates. Various types of carbohydrates and sources of carbohydrates are contemplated. The term “carbohydrates” refers to polyhydroxy aldehydes, ketones, alcohols, acids, their simple derivatives and their polymers having linkages of the acetal type. Carbohydrates are a type of macronutrients with a range of physical and physiological properties and health benefits. Preferably, compositions of the invention include more than one type of carbohydrate (i.e. more than one chemically defined substance). Classification of carbohydrates is based on their chemistry, preferably by molecular size (as determined by degree of polymerization (DP)), the type of linkage (e.g. a or non-α) and/or character of individual monomers. See, for example, FAO (1998) Carbohydrates in human nutrition. Food and Agriculture Organization of the United Nations: Rome. Report of a Joint FAO/WHO Expert Consultation. FAO Food and Nutrition Paper no. 66.

When carbohydrates are classified according to their degree of polymerization, they may be divided initially into three principal groups, namely sugars, oligosaccharides and polysaccharides. Various ratios of sugars, oligosaccharides and polysaccharides are envisioned. In some embodiments, a single dosage form composition of the invention comprises only sugars. In other embodiments, a composition of the invention comprises only oligosaccharides. In other embodiments, a single dosage form composition of the invention comprises only polysaccharides. In other embodiments, a single dosage form composition of the invention comprises sugars and oligosaccharides. In other embodiments, a single dosage form composition of the invention comprises sugars and polysaccharides. In other embodiments, a single dosage form composition of the invention comprises oligosaccharides and polysaccharides. In other embodiments, a single dosage form composition of the invention comprises sugars, oligosaccharides and polysaccharides. When sugars, oligosaccharides and/or polysaccharides are present in the above embodiments, the amount of each group may be at least about 10%, 20%, 30%, 40%, 50%, 60%, 70%, 80%, 90% by weight of the total amount of carbohydrates, provided the sum of all three groups does not exceed 100%.

As used herein, the term “sugar” refers to monosaccharides, disaccharides, and polyols. A monosaccharide is the simplest carbohydrate in that it cannot be hydrolyzed to smaller carbohydrates. In certain embodiments, monosaccharides may be further characterized by number of carbon atoms—e.g. diose (2 carbon atoms), triose (3 carbon atoms), tetrose (4 carbon atoms), pentose (5 carbon atoms), hexose (6 carbon atoms), heptose (seven carbon atoms), etc. Non-limiting examples of suitable monosaccharides include deoxyribose, fructose, galactose, glucose, lyxose, mannose, ribose, ribulose, xylose, and xylulose. A disaccharide is composed of two monosaccharide units bound together by a glycosidic linkage. Non-limiting examples of suitable disaccharides include inulobiose, lactose, maltose, sucrose and trehalose. A polyol is a sugar alcohol. Non-limiting examples of suitable polyols include erythritol, isomalt, lactitol, maltitol, mannitol, sorbitol, and xylitol. Monosaccharides and disaccharides may be produced from the hydrolysis of starch and/or other non-starch polysaccharides, and/or they may be produced synthetically.

An oligosaccharide is composed of three to nine monosaccharide units, each unit bound to the next by a glycosidic linkage; and a polysaccharide is composed of ten or more monosaccharide units, each unit bound to the next by a glycosidic linkage. Oligosaccharides and polysaccharides may be branched or linear, may be comprised of α- and/or β-glycosidic bonds, and may be comprised of one or more types of monosaccharides. Oligosaccharides and polysaccharides may be produced synthetically. Oligosaccharides may also be produced from the partial hydrolysis of starch and/or other non-starch polysaccharides.

Non-limiting examples of suitable oligosaccharides include dextrans (e.g. maltodextrin, cyclodextrin, β-limit dextran, α-limit dextran, polydextrin), fructo-oligosaccharides (e.g. inulin), galacto-oligosaccharides, mannan-oligosaccharides, arabino-oligosaccharides, and alpha-galactosyl derivatives of sucrose (e.g. raffinose, stachyose, verbascose).

In certain embodiments, polysaccharides may be further characterized as starches and non-starch polysaccharides. Starch is an α-glucan and, therefore, consists of glucose molecules linked with α-glycosidic bonds. Starch may be comprised of amylose, amylopectin, or combinations thereof. Included within the definition of “starch” are both “unmodified starch” and “modified starch.” Unmodified starch refers to starch that has all its native characteristics. Modified starch refers to starch that has one or more of its native characteristics altered by treatment in accordance with good manufacturing practice. Non-limiting types of treatment include heating, acid treatment, alkaline treatment, enzyme treatment, oxidation, substitution (i.e. introduction of side groups, for example, by etherification, esterification, or other methods known in the art), and cross-linking. Non-limiting examples of suitable starch include cane syrup, corn syrup, high fructose corn syrup, dextrans (e.g. amylodextrin, maltodextrin, cyclodextrin, β-limit dextran, α-limit dextran, polydextrin), and tapioca syrup.

Non-starch polysaccharides (NSPs) are non-α-glucan polysaccharides. Many, though not all, NSPs are found in plant cell walls. Non-limiting examples of suitable NSPs include beta-glucans, inulin, cellulose, hemicellulose, pectin, plant gum and mucilages (e.g. acacia gum, karaya, guar gum, carob gum, locust bean gum, konjac, xanthan, and tragacanth), and algal polysaccharides (e.g. carageenan, agar, and alginate), and arabinogalactan (plant or microbial).

In some embodiments, a single dosage form composition of the invention comprises at least about 5 g of carbohydrates. For example, a single dosage form composition of the invention may comprise at least about 5 g, at least about 10 g, at least about 15 g, at least about 20 g, at least about 25 g, at least about 30 g, at least about 35 g, at least about 40 g, at least about 45 g, at least about 50 g, at least about 55 g, or at least about 60 g of carbohydrates.

In other embodiments, a single dosage form composition of the invention comprises about 5 g to about 120 g of carbohydrates. In other embodiments, a single dosage form composition of the invention comprises about 5 g to about 115 g of carbohydrates or about 10 g to about 120 g of carbohydrates. In other embodiments, a single dosage form composition of the invention comprises about 5 g to about 110 g of carbohydrates, about 10 g to about 115 g of carbohydrates, or about 15 g to about 120 g of carbohydrates. In other embodiments, a single dosage form composition of the invention comprises about 5 g to about 105 g of carbohydrates, about 10 g to about 110 g of carbohydrates, about 15 g to about 115 g of carbohydrates, or about 20 g to about 120 g of carbohydrates. In other embodiments, a single dosage form composition of the invention comprises about 5 g to about 100 g of carbohydrates, about 10 g to about 105 g of carbohydrates, about 15 g to about 110 g of carbohydrates, about 20 g to about 115 g of carbohydrates, or about 20 g to about 120 g of carbohydrates. In other embodiments, a single dosage form composition of the invention comprises about 5 g to about 50 g of carbohydrates, about 10 g to about 55 g of carbohydrates, about 15 g to about 60 g of carbohydrates, or about 20 g to about 65 g of carbohydrates. In other embodiments, a single dosage form composition of the invention comprises about 5 g to about 20 g of carbohydrates, about 10 g to about 25 g of carbohydrates, about 15 g to about 30 g of carbohydrates, or about 20 g to about 35 g of carbohydrates. In other embodiments, a single dosage form composition of the invention comprises about 25 g to about 40 g of carbohydrates, about 30 g to about 45 g of carbohydrates, or about 35 g to about 50 g of carbohydrates.

Suitable methods for analyzing the carbohydrate content of compositions of the invention are known in the art. The following is included to demonstrate some of the known methods, but it will be appreciated by those of skill in the art that the techniques disclosed are not limiting. Further details may be found, for example, in Southgate, D. A. T. 1991. Determination of food carbohydrates. Elsevier Science Publishers, Ltd., Barking; and Englyst K, et al. (2007). “Nutritional characterisation and measurement of dietary carbohydrates.” Eur J Clin Nutr 61 (Suppl 1), S19-S39.

Monosaccharides and disaccharides can be analyzed specifically by enzymatic, gas-liquid chromatography (GLC) or high performance liquid chromatography (HPLC) methods. Depending on the composition to be analyzed, extraction of the low molecular weight carbohydrates in aqueous ethanol, usually 80% (v/v), may occur before analysis. Enzymatic methods may be preferable when one single carbohydrate is to be analyzed, e.g. glucose, as the end point of starch analysis. When several different monosaccharides are to be determined simultaneously, HPLC or GLC methods are preferable. HPLC methods can also be used to measure polyols.

Oligosaccharide analysis of a composition can also be determined by GLC or HPLC. These methods work well for purified preparations, but in complex compositions, enzymatic hydrolysis and determination of liberated monosaccharides is an alternative for specific determination.

Quantitative analysis of starch in foods may occur by enzymatic degradation and specific determination of liberated glucose. The determination of NSP is based on the following steps: (a) degradation of starch by enzymatic hydrolysis after solublization, (b) removal of low molecular weight carbohydrates, including starch hydrolysis products, (c) hydrolysis of the NSP to their constituent monomers, and (d) quantitative determination of those monomers. Non-limiting examples of method for specific determination of the liberated monomers include GLC with alditol acetate derivatives, HPLC detection, and colourimetric determination. Alternatively or in addition, fractions of NSP, such as cellulose and non-cellulosic polysaccharides, can be separated by using sequential extraction and hydrolysis methods known in the art.

### (b) Proteins

A single dosage form composition of the invention comprises protein. Various protein sources are contemplated including animal and plant proteins, as well as synthetic proteins. Also contemplated are both complete and incomplete proteins, though complete proteins or complementary incomplete proteins are preferred. As used herein, a “complete protein” refers to a protein that provides an adequate proportion of all of the essential amino acids necessary for the dietary needs of a subject, an “incomplete protein” refers to a protein that is low in one or more essential amino acid necessary for the dietary needs of a subject, and “complementary incomplete proteins” refers to two or more proteins that together provide an adequate amount of all the essential amino acids necessary for the dietary needs of a subject. Non-limiting examples of suitable proteins includes caseinates, milk proteins (e.g. whey, casein, lactoferrin, glycomacropeptide), egg proteins, buckwheat proteins, quinoa proteins, spelt proteins, soy proteins, rice proteins, hemp proteins, pea proteins, beef proteins, poultry proteins, fish proteins, cranberry proteins, artichoke proteins, as well as isolates, concentrates, agglomerates and hydrolysates thereof. Proteins may also be provided in the form of plant derived butters (e.g. almond butter, apple butter, cashew butter, cocoa butter, coconut butter, hazelnut butter, peanut butter, tahini). Combinations of suitable proteins are also contemplated.

The current Recommended Dietary Allowance (RDA) for protein varies by age. Generally, however, for humans fourteen years of age or older, the RDA is 0.8 g per kilogram of body weight, or approximately 46-56 gm/day (Institute of Medicine (2002) Dietary Recommended Intakes: Energy, Carbohydrate, Fiber, Fat, Fatty Acids, Cholesterol, Protein and Amino Acids 2002 Institute of Medicine, National Academy Press Washington, DC; at page 589 and 645). No additional dietary protein is suggested for healthy adults undertaking resistance or endurance exercise (Institute of Medicine (2002) Dietary Recommended Intakes: Energy, Carbohydrate, Fiber, Fat, Fatty Acids, Cholesterol, Protein and Amino Acids 2002, Institute of Medicine, National Academy Press Washington, DC; at page 661.) In contrast to the recommendations by the Institute of Medicine, applicants have discovered protein supplementation is beneficial to athletes, in particular when provided in combination with carbohydrates in the ratio and amounts disclosed herein. Moderately intense training athletes may consume about 1 to about 1.5 grams/kg/day of protein, while athletes involved in high volume intense training may consume about 1.5 to about 2.0 grams/kg/day of protein, or more.

In some embodiments, a single dosage form composition of the invention comprises at least about 10 g of proteins. For example, a single dosage form composition of the invention may comprise at least about 10 g, at least about 15 g, at least about 20 g, at least about 25 g, at least about 30 g, at least about 35 g, at least about 40 g, at least about 45 g, at least about 50 g, at least about 55 g, or at least about 60 g of proteins.

In other embodiments, a single dosage form composition of the invention comprises about 10 g to about 60 g of proteins. In other embodiments, a single dosage form composition of the invention comprises about 10 g to about 55 g of proteins or about 15 g to about 60 g of proteins. In other embodiments, a single dosage form composition of the invention comprises about 10 g to about 50 g of proteins, about 15 g to about 55 g of proteins, or about 20 g to about 60 g of proteins. In other embodiments, a single dosage form composition of the invention comprises about 10 g to about 45 g of proteins, about 15 g to about 50 g of proteins, about 20 g to about 55 g of proteins, or about 25 g to about 60 g of proteins. In other embodiments, a single dosage form composition of the invention comprises about 10 g to about 40 g of proteins, about 15 g to about 45 g of proteins, about 20 g to about 50 g of proteins, about 25 g to about 55 g of proteins, or about 30 g to about 60 g of proteins. In other embodiments, a single dosage form composition of the invention comprises about 10 g to about 30 g of proteins, about 20 g to about 40 g of proteins, about 30 g to about 50 g of proteins, or about 40 g to about 60 g of proteins. In other embodiments, a single dosage form composition of the invention comprises about 10 g to about 20 g of proteins, about 20 g to about 30 g of proteins, or about 30 g to about 40 g of proteins. In other embodiments, a single dosage form composition of the invention comprises about 30 g to about 40 g of proteins, about 40 g to about 50 g of proteins, or about 50 g to about 60 g of proteins.

Protein and amino acid requirements go hand-in-hand, as proteins are an important source of amino acids. Branched-chain amino acids, e.g. leucine, isoleucine, and valine, can stimulate the building of protein in muscle, reduce muscle breakdown, improve endurance, and reduce fatigue. The RDA of branched-chain amino acids is 85 mg/kg/day for adults—leucine 42 mg/kg/day, isoleucine 19 mg/kg/day, valine 24 mg/kg/day (Institute of Medicine (2002) Dietary Recommended Intakes: Energy, Carbohydrate, Fiber, Fat, Fatty Acids, Cholesterol, Protein and Amino Acids 2002 Institute of Medicine, National Academy Press Washington, DC; at page 707). Different sources of protein provide varying amounts of branched chain amino acids. A single dosage form composition of the invention preferably provides at least 3.5 g of branched-chain amino acids, at least 4.0 g of branched-chain amino acids, at least 4.5 g of branched-chain amino acids, at least 5.0 g of branched-chain amino acids, or at least 5.5 g of branched-chain amino acids. The amount of branched-chain amino acids in a composition.

Methods for determining overall protein content in a composition are known in the art. Suitable methods include, but are not limited to, the Kjeldahl method, the enhanced Dumas method, ultraviolet light absorption at 280 nm, the Biuret method, and the Lowry method.

### (c) Magnesium

In certain embodiments, a single dosage form composition of the invention comprises magnesium. Magnesium compounds and complexes suitable for consumption are known in the art and may be used in a single dosage form composition of the invention. Non-limiting examples of suitable magnesium compounds and complexes that may be used in a single dosage form composition of the invention include magnesium amino acid chelates, complexes or admixtures (e.g. magnesium aspartate, magnesium carnitine chelate, magnesium creatine chelate, magnesium glycinate chelate, magnesium glycyl glutamine chelate, magnesium lysyl glycinate, magnesium taurate, magnesium threonate), magnesium carbonate, magnesium chloride, magnesium citrate, magnesium lactate, dimagnesium malate, magnesium oxide, and magnesium sulfate. Mixtures of two or more of the above magnesium compounds and/or complexes are also contemplated.

Various amounts magnesium compounds and complexes are envisioned. A skilled artisan will appreciate the amount of elemental magnesium will vary depending on the source. Accordingly, the amount of magnesium expressed herein refers to the amount of elemental magnesium and suitable amounts of the appropriate magnesium compound(s) and/or complexes may be calculated therefrom. Generally, the amount of magnesium when present in the single dosage form composition is at least about 400 mg. For example, a single dosage form composition of the invention may comprise at least about 400 mg, at least about 425 mg, at least about 450 mg, at least about 500 mg, at least about 525 mg, at least about 550 mg, at least about 575 mg, at least about 600 mg, at least about 625 mg, at least about 650 mg, at least about 675 mg, at least about 700 mg, at least about 725 mg, at least about 750 mg, at least about 775 mg, at least about 800 mg, at least about 825 mg, at least about 850 mg, at least about 875 mg, at least about 900 mg, at least about 925 mg, at least about 950 mg, at least about 975 mg, at least about 1000 mg of magnesium, at least about 1025 mg, at least about 1050 mg, at least about 1075 mg, at least about 1100 mg, at least about 1125 mg, at least about 1150 mg, at least about 1175 mg, at least about 1200 mg, at least about 1225 mg, at least about 1250 mg, at least about 1275 mg, at least about 1300 mg, at least about 1325 mg, at least about 1350 mg, at least about 1375 mg, at least about 1400 mg, at least about 1425 mg, at least about 1450 mg, at least about 1475 mg, at least about 1500 mg, at least about 1525 mg, at least about 1550 mg, or at least about 1575 mg of magnesium. In preferred embodiments, the amount of magnesium present in a single dosage form composition of the invention is no greater than about 1600 mg.

In some embodiments, a single dosage form composition of the invention comprises about 400 mg to about 1600 mg of magnesium. In other embodiments, a single dosage form composition of the invention comprises about 500 mg to about 1500 mg of magnesium or about 500 mg to about 1600 mg of magnesium. In other embodiments, a single dosage form composition of the invention comprises about 400 mg to about 1400 mg of magnesium, about 500 mg to about 1500 mg of magnesium, or about 600 mg to about 1600 mg. In other embodiments, a single dosage form composition of the invention comprises about 400 mg to about 1300 mg of magnesium, about 500 mg to about 1400 mg of magnesium, about 600 mg to about 1500 mg, or about 700 mg to about 1600 mg of magnesium. In other embodiments, a single dosage form composition of the invention comprises about 400 mg to about 1200 mg of magnesium, about 500 mg to about 1300 mg of magnesium, about 600 mg to about 1400 mg, about 700 mg to about 1500 mg of magnesium, or about 800 mg to about 1600 mg of magnesium. In other embodiments, a single dosage form composition of the invention comprises about 400 mg to about 1100 mg of magnesium, about 500 mg to about 1200 mg of magnesium, about 600 mg to about 1300 mg, about 700 mg to about 1400 mg of magnesium, about 800 mg to about 1500 mg of magnesium, or about 900 mg to about 1600 mg of magnesium.

In certain embodiments, a single dosage form composition of the invention comprises about 400 mg to about 1000 mg of magnesium, preferably about 500 mg to about 900 mg of magnesium, more preferably about 500 mg to about 800 mg of magnesium.

Alternatively, a single dosage form composition of the invention comprises about 400 mg to about 800 mg of magnesium, preferably about 450 mg to about 750 mg of magnesium, more preferably about 500 mg to about 700 mg of magnesium.

In yet another alternative, a single dosage form composition of the invention comprises about 600 mg to about 1000 mg of magnesium, preferably about 650 mg to about 950 mg of magnesium, more preferably about 700 mg to about 900 mg of magnesium.

Methods for determining the amount of magnesium in a composition are known in the art. Non-limiting examples include atomic absorption spectrometry, colorimetry, and precipitation and extraction. A detailed discussion may be found, for example, in “Food Composition Data: Production, Management and Use,” Second edition, by Greenfield and Southgate, Food and Agricultural Organization of the United Nations, Rome 2003.

### (d) Zinc

In certain embodiments, a single dosage form composition of the invention comprises zinc. Zinc compounds and complexes suitable for consumption are known in the art and contemplated herein. Non-limiting examples of suitable zinc compounds and complexes that may be used in a single dosage form composition of the invention include zinc oxide; zinc sulfate; zinc amino acid chelates, complexes or admixtures (e.g., zinc arginate, zinc aspartate, zinc bisglycinate, citrated zinc bisglycinate, zinc carnitine chelate, zinc creatine chelate, and zinc histidinate); zinc acetate; zinc acetate dihydrate; zinc ascorbate; zinc carbonate; zinc chloride; zinc citrate; zinc gluconate; zinc ketoglutarate; zinc malate; zinc picolinate; zinc stearate; zinc succinate; and zinc undecylenate. Mixtures of two or more of the above zinc compounds and/or complexes are also contemplated.

Various amounts zinc compounds and complexes are envisioned. A skilled artisan will appreciate the amount of elemental zinc will vary depending on the source. Accordingly, the amount of zinc expressed herein refers to the amount of elemental zinc and suitable amounts of the appropriate zinc compound(s) and/or complexes may be calculated therefrom. Generally, the amount of zinc when present in the single dosage form composition is at least about 1 mg. For example, a single dosage form composition of the invention may comprise at least about 1 mg, at least about 5 mg, at least about 10 mg, at least about 15 mg, at least about 20 mg, at least about 25 mg, at least about 30 mg, at least about 35 mg, at least about 40 mg, at least about 45 mg, at least about 50 mg, at least about 55 mg, at least about 60 mg, at least about 70 mg, at least about 75 mg, at least about 80 mg, at least about 85 mg, at least about 90 mg, at least about 95 mg, at least about 100 mg, at least about 105 mg, at least about 110 mg, at least about 115 mg, at least about 120 mg, at least about 125 mg, at least about 130 mg, at least about 135 mg, at least about 140 mg, or at least about 145 mg of zinc. In preferred embodiments, the amount of zinc present in a single dosage form composition of the invention is no greater than about 150 mg.

In some embodiments, a single dosage form composition of the invention comprises about 1 mg to about 150 mg of zinc. In other embodiments, a single dosage form composition of the invention comprises about 1 mg to about 125 mg of zinc or about 25 mg to about 150 mg of zinc. In other embodiments, a single dosage form composition of the invention comprises about 1 mg to about 100 mg of zinc, about 25 mg to about 125 mg of zinc, or about 50 mg to about 150 mg. In other embodiments, a single dosage form composition of the invention comprises about 1 mg to about 75 mg of zinc, about 25 mg to about 100 mg of zinc, about 50 mg to about 125 mg, or about 75 mg to about 150 mg of zinc. In other embodiments, a single dosage form composition of the invention comprises about 1 mg to about 50 mg of zinc, about 25 mg to about 75 mg of zinc, about 50 mg to about 100 mg, about 75 mg to about 125 mg of zinc, or about 100 mg to about 150 mg of zinc.

In certain embodiments, a single dosage form composition of the invention comprises about 1 mg to about 50 mg of zinc, preferably about 5 mg to about 45 mg of zinc, more preferably about 10 mg to about 40 mg of zinc.

Alternatively, a single dosage form composition of the invention comprises about 5 mg to about 45 mg of zinc, preferably about 5 mg to about 35 mg of zinc, more preferably about 5 mg to about 25 mg of zinc.

In yet another alternative, a single dosage form composition of the invention comprises about 10 mg to about 40 mg of zinc, preferably about 10 mg to about 30 mg of zinc, more preferably about 10 mg to about 20 mg of zinc.

Methods for determining the amount of zinc in a composition are known in the art. Non-limiting examples include atomic absorption spectrometry, colorimetry, and precipitation and extraction. A detailed discussion may be found, for example, in “Food Composition Data: Production, Management and Use,” Second edition by Greenfield and Southgate, Food and Agricultural Organization of the United Nations, Rome 2003.

### (e) Choline

In certain embodiments, a single dosage form composition of the invention comprises choline in a form of one or more physiologically acceptable salts, phospholipid bound choline, choline precursors and choline metabolites, wherein the choline precursors or choline metabolites are capable of being converted into choline, and in an amount that provides at least about 10 mg to about 3000 mg of choline (i.e (2-(hydroxyethyl)trimethylammonium). Calculation of suitable amounts of choline salts, phospholipid bound forms, intermediates of choline and derivatives of choline to provide at least 10 mg to about 3000 mg of choline is well within the level of one of ordinary skill in the art.

The RDA for choline for a male human subjects age 19+ years is 550 mg per day; and the RDA for choline for a female human subject age 19+ years is 425 mg per day (Institute of Medicine (2002) Dietary Recommended Intakes: Energy, Carbohydrate, Fiber, Fat, Fatty Acids, Cholesterol, Protein and Amino Acids 2002 Institute of Medicine, National Academy Press Washington, DC; at page 1320). Recently, the Food and Drug Administration established a Reference Dietary Intake (RDI) of 550 mg of choline for adults and children that are 4 years of age or older (§ 101.9(c)(8)(iv), see also Federal Register, May 27, 2016, at page 33905). Thus, compositions of the invention comprising at least about 10 mg to about 3000 mg of choline may provide an amount of choline that is in excess of a subject's RDI/RDA, depending in some instances upon that subject's diet.

Choline is a multi-tasking nutrient, important for cell structure and function, lipid metabolism, neurotransmission, cell signaling and genetic regulation. For example, maintenance of the nervous system during intense exercise is necessary to drive the continued neural activation of muscles and to delay the onset of so-called “central fatigue,” which is associated with long periods of sustained muscular usage. Decreased choline (and consequently, acetylcholine) may be associated with delays in transmission of muscle contraction impulses. In states of dietary choline deprivation and metabolic stress such as extreme exertion, membrane phospholipids (phosphatidylcholine and sphingomyelin) may be catabolized by phospholipase enzymes in an effort to maintain levels of brain choline and provide for its release into synaptic clefts in the hippocampus and the neuromuscular junction. Choline intake is necessary to regulate the activity of the various enzymes that synthesize, break down and influence the release of the neurotransmitter acetylcholine, with a secondary benefit of helping in the retention of cell membrane integrity. Choline also plays a role in mobilization of fat, which can serve as a supplemental fuel source during exercise. Choline administration may also help reduce plasma levels of homocysteine a harmful metabolic by-product. This in turn supports creatine biosynthesis and enhances muscle recovery. As further evidence of the above, the European Food Safety Authority (EFSA) has approved the following health claims for choline (82.5 mg per dosage form): choline contributes to normal homocysteine metabolism, choline contributes to normal lipid metabolism, and choline contributes to maintenance of normal liver function (ec.europa.eu/nuhclaims/).

Accordingly, a single dosage form composition may comprise a choline amount of about 10 mg to about 3000 mg, about 10 mg to about 2500 mg, about 10 mg to about 2000 mg, about 10 mg to about 1500 mg, about 10 mg to about 1000 mg, about 10 mg to about 500 mg, or about 10 mg to about 250 mg. Further, a single dosage form composition may comprise a choline amount of about 25 mg to about 3000 mg, about 25 mg to about 2500 mg, about 25 mg to about 2000 mg, about 25 mg to about 1500 mg, about 25 mg to about 1000 mg, about 25 mg to about 500 mg, or about 25 mg to about 250 mg. Still further, a single dosage form composition may comprise a choline amount of about 50 mg to about 3000 mg, about 50 mg to about 2500 mg, about 50 mg to about 2000 mg, about 50 mg to about 1500 mg, about 50 mg to about 1000 mg, about 50 mg to about 500 mg, or about 50 mg to about 250 mg. Alternatively, a single dosage form composition may comprise a choline amount of about 100 mg to about 3000 mg, about 100 mg to about 2500 mg, about 100 mg to about 2000 mg, about 100 mg to about 1500 mg, about 100 mg to about 1000 mg, about 100 mg to about 500 mg, or about 100 mg to about 250 mg. Further, a single dosage form composition may comprise a choline amount of about 200 mg to about 3000 mg, about 200 mg to about 2500 mg, about 200 mg to about 2000 mg, about 200 mg to about 1500 mg, about 200 mg to about 1000 mg, or about 200 mg to about 500 mg. In addition, a single dosage form composition may comprise a choline amount of about 250 mg to about 3000 mg, about 250 mg to about 2500 mg, about 250 mg to about 2000 mg, about 250 mg to about 1500 mg, about 250 mg to about 1000 mg, about 250 mg to about 750 mg, or about 250 mg to about 500 mg. A single dosage form composition may also comprise a choline amount of about 350 mg to about 3000 mg, about 350 mg to about 2500 mg, about 350 mg to about 2000 mg, about 350 mg to about 1500 mg, about 350 mg to about 1000 mg, about 350 mg to about 750 mg, or about 350 mg to about 500 mg. A single dosage form composition may also comprise a choline amount of about 500 mg to about 3000 mg, about 500 mg to about 2500 mg, about 500 mg to about 2000 mg, about 500 mg to about 1500 mg, about 500 mg to about 1000 mg, or about 500 mg to about 750 mg.

Choline salts comprises the chemical formula (CH₃)₃N⁺(CH₂)₂OHX⁻, wherein X⁻ is a negative counterion. Non-limiting examples of choline salts include choline bitartrate, choline chloride, choline dihydrogen citrate, choline salicylate, choline phosphate, choline bicarbonate, and choline magnesium trisalicylate. Specifically, a single dosage form composition of the disclosure may comprise choline bitartrate, choline dihydrogen citrate, or choline chloride. Preferably, the choline salt has a choline cation concentration that is greater than 40% by weight, greater than 50% by weight, greater than 60% by weight, greater than 70% by weight, greater than 75% by weight, or greater than 80% by weight. Alternatively, the form of choline used in a single dosage form composition of the disclosure may comprise a phospholipid bound choline including, but not limited to, phosphatidylcholine and soy lecithin. In addition, choline precursors and choline metabolites (e.g. compounds capable of being converted into choline) such as CDP-choline (also known as citicoline, cytidine diphosphate-choline or cytidine 5′-diphosphocholine) may be used in a single dosage form composition of the disclosure.

In a particular embodiment, choline is present in the form of choline bitartrate. Bitartrate contains two chiral carbons. Accordingly, bitartrate may be DL-choline bitrartate, D-choline bitartrate or L-choline bitartrate. In the D- and L-forms, both chiral carbons are either D or L, respectively. In certain embodiments, the choline bitartrate is L-choline bitartrate. Accordingly, the L-choline bitartrate is optically active to plane polarizing light. More specifically, the L-choline bitartrate rotates plane polarized light more than +17.5 degrees. Only L-choline bitartrate is in the USP monograph. As L-amino acids are generally found in nature, the L-form of choline bitartrate may also be referred to as the natural form of choline bitartrate. In other embodiment, the choline bitartrate is a racemic mixture of D-choline bitartrate, L-choline bitartrate or DL-choline bitartrate. Accordingly, the racemic mixture is optically inactive to plane polarizing light. More specifically, the racemic mixture rotates plane polarized light less than +17.5 degrees.

In another particular embodiment, choline is present in the form of choline chloride as a product that (a) contains a choline cation concentration that is at least about 60% by weight, at least about 70% by weight, or at least about 75% by weight, (b) has a moisture content below 2.5%, preferably about 1%, more preferably about 0.5%, and (c) is substantially free of organic solvent (e.g. the wt % of the solvent is ≤25%, preferably ≤20%, more 15%, even more preferably ≤10%). Although all choline chloride is the same at the molecular level, there are quantifiable differences between commercially available choline chloride products. For example, a choline chloride product may contain chloride salts (e.g. KCl, MgCl₂, NH₄Cl, etc.) in addition to choline chloride. As a result, tests that confirm choline chloride content by assuming a 1:1 ratio between choline content and chloride content and quantifying the chloride content will overestimate the choline content due to the presence of excess chloride ions. For accurate confirmation of choline chloride content, the Reinecke salt test is often recommended with ion chromatography as a final confirmation. Both offer high levels of accuracy and precisely identify any product adulteration. The choice of manufacturing process may also result in measurable differences in a choline chloride product such as varying amounts of residual total trimethylaminesammonium, dioxin content, and moisture content. In an exemplary embodiment, choline is present in the form of choline chloride, for example as the product Vitacholine™.

### (f) Creatine

A single dosage form composition of the invention of may further comprise creatine. Suitable forms of creatine are known in the art and contemplated herein. Non-limiting examples of suitable forms of creatine include creatine monohydrate, creatine phosphate, and a metal creatine chelate, complex or admixture. The metal creatine chelate, complex or admixture can have a metal chosen from magnesium, calcium, copper, zinc, iron, chromium, cobalt, molybdenum, selenium, and manganese. Mixtures of two or more of the above forms of creatine are also contemplated.

Various amounts of creatine compounds and complexes are envisioned. A skilled artisan will appreciate the amount of elemental creatine will vary depending on the source. Accordingly, the amount of creatine expressed herein refers to the amount of elemental creatine and suitable amounts of the appropriate creatine compound(s) and/or complexes may be calculated therefrom. Generally, the amount of creatine when present in the single dosage form composition is at least about 0.1 g. For example, a single dosage form composition of the invention may comprise at least about 0.1 g, at least about 0.5 g, at least about 1 g, at least about 1.5 g, at least about 2 g, at least about 2.5 g, at least about 3.0 g, at least about 3.5 g, at least about 4 g, at least about 4.5 g, at least about 5 g, at least about 5.5 g, at least about 6 g, at least about 7 g, at least about 7.5 g, at least about 8 g, at least about 8.5 g, at least about 9 g, at least about 9.5 g, at least about 10 g, at least about 10.5 g, at least about 11 g, at least about 11.5 g, at least about 12 g, at least about 12.5 g, at least about 13 g, at least about 14 g, at least about 15 g of creatine, at least about 16 g of creatine, at least about 17 g of creatine, at least about 18 g of creatine, at least about 19 g of creatine, at least about 20 g of creatine, at least about 21 g of creatine, at least about 22 g of creatine, at least about 23 g of creatine, at least about 24 g of creatine, or at least about 25 g of creatine. In preferred embodiments, the amount of creatine present in a single dosage form composition of the invention is no greater than about 25 g.

In some embodiments, a single dosage form composition of the invention comprises about 0.1 g to about 25 g of creatine. In other embodiments, a single dosage form composition of the invention comprises about 0.1 g to about 20 g of creatine or about 0.5 g to about 25 g of creatine. In other embodiments, a single dosage form composition of the invention comprises about 0.1 g to about 15 g of creatine, about 0.5 g to about 20 g of creatine, or about 1 g to about 25 g. In other embodiments, a single dosage form composition of the invention comprises about 0.1 g to about 10 g of creatine, about 0.5 g to about 15 g of creatine, about 1 g to about 20 g, or about 5 g to about 25 g of creatine. In other embodiments, a single dosage form composition of the invention comprises about 0.1 g to about 5 g of creatine, about 0.5 g to about 10 g of creatine, about 1 g to about 15 g, about 5 g to about 20 g of creatine, or about 10 g to about 25 g of creatine.

In certain embodiments, a single dosage form composition of the invention comprises about 0.1 g to about 20 g of creatine, preferably about 0.5 g to about 15 g of creatine, more preferably about 1 g to about 12.5 g of creatine.

Alternatively, a single dosage form composition of the invention comprises about 1 g to about 20 g of creatine, preferably about 1 g to about 15 g of creatine, more preferably about 1 g to about 10 g of creatine.

In yet another alternative, a single dosage form composition of the invention comprises about 5 g to about 15 g of creatine, preferably about 5 g to about 12.5 g of creatine, or more preferably about 5 g to about 10 g of creatine.

In yet another alternative, a single dosage form composition of the invention comprises about 1 g to about 10 g of creatine, preferably about 2.5 g to about 7.5 g of creatine, or more preferably about 3 g to about 6 g of creatine.

### (g) Carnitine

A single dosage form composition of the invention of may further comprise carnitine. Suitable forms of carnitine are known in the art and contemplated herein. Non-limiting examples of suitable forms of carnitine include L-carnitine and its salts, acetyl-L-carnitine, and a metal carnitine chelate, complex or admixture. The metal carnitine chelate, complex or admixture can have a metal chosen from magnesium, calcium, copper, zinc, iron, chromium, cobalt, molybdenum, selenium, and manganese. Mixtures of two or more of the above forms of carnitine are also contemplated. In an exemplary embodiment, Carnipure® may be used.

Various amounts carnitine compounds and complexes are envisioned. A skilled artisan will appreciate the amount of elemental carnitine will vary depending on the source. Accordingly, the amount of carnitine expressed herein refers to the amount of elemental carnitine and suitable amounts of the appropriate carnitine compound(s) and/or complexes may be calculated therefrom. Generally, the amount of carnitine when present in the single dosage form composition is at least about 0.1 g. For example, a single dosage form composition of the invention may comprise at least about 0.1 g, at least about 0.5 g, at least about 1 g, at least about 1.5 g, at least about 2 g, at least about 2.5 g, at least about 3.0 g, at least about 3.5 g, at least about 4 g, at least about 4.5 g, at least about 5 g, at least about 5.5 g, at least about 6 g, at least about 7 g, at least about 7.5 g, at least about 8 g, at least about 8.5 g, at least about 9 g, at least about 9.5 g, at least about 10 g, at least about 10.5 g, at least about 11 g, at least about 11.5 g, at least about 12 g, at least about 12.5 g, at least about 13 g, at least about 14 g, at least about 15 g of carnitine, at least about 16 g of carnitine, at least about 17 g of carnitine, at least about 18 g of carnitine, at least about 19 g of carnitine, at least about 20 g of carnitine, at least about 21 g of carnitine, at least about 22 g of carnitine, at least about 23 g of carnitine, at least about 24 g of carnitine, or at least about 25 g of carnitine. In preferred embodiments, the amount of carnitine present in a single dosage form composition of the invention is no greater than about 25 g.

In some embodiments, a single dosage form composition of the invention comprises about 0.1 g to about 25 g of carnitine. In other embodiments, a single dosage form composition of the invention comprises about 0.1 g to about 20 g of carnitine or about 0.5 g to about 25 g of carnitine. In other embodiments, a single dosage form composition of the invention comprises about 0.1 g to about 15 g of carnitine, about 0.5 g to about 20 g of carnitine, or about 1 g to about 25 g. In other embodiments, a single dosage form composition of the invention comprises about 0.1 g to about 10 g of carnitine, about 0.5 g to about 15 g of carnitine, about 1 g to about 20 g, or about 5 g to about 25 g of carnitine. In other embodiments, a single dosage form composition of the invention comprises about 0.1 g to about 5 g of carnitine, about 0.5 g to about 10 g of carnitine, about 1 g to about 15 g, about 5 g to about 20 g of carnitine, or about 10 g to about 25 g of carnitine.

In certain embodiments, a single dosage form composition of the invention comprises about 0.1 g to about 10 g of carnitine, preferably about 0.5 g to about 9.5 g of carnitine, more preferably about 1 g to about 9 g of carnitine.

Alternatively, a single dosage form composition of the invention comprises about 1 g to about 10 g of carnitine, preferably about 1 g to about 7.5 g of carnitine, more preferably about 1 g to about 5 g of carnitine.

In yet another alternative, a single dosage form composition of the invention comprises about 2.5 g to about 12.5 g of carnitine, preferably about 2.5 g to about 10 g of carnitine, more preferably about 2.5 g to about 7.5 g of carnitine.

### (h) Fats

A single dosage form composition of the invention may further comprise fats. Suitable forms of fats are known in the art and contemplated herein. Fats may be modified or in their natural state. Fats may also be synthetic. Non-limiting examples of suitable fats include plant derived oils (e.g. canola oil, coconut oil, corn oil, flaxseed oil, hemp oil, olive oil, palm oil, peanut oil, safflower oil, soybean oil, sunflower oil, wheat germ oil), and fats obtained from plant derived butters (e.g. almond butter, apple butter, cashew butter, cocoa butter, coconut butter, hazelnut butter, peanut butter, tahini). Suitable fats also include short chain triglycerides, medium chain triglycerides, long chain triglycerides, phospholipids, short chain fatty acids and medium chain fatty acids. Trace and or small (0.01-5 g) amounts of fats may also come from proteins that are added to the composition. Mixtures of two or more of the above fats are also contemplated.

In some embodiments, a single dosage form composition of the invention comprises a weight ratio of proteins to fat that is about 10:0.1 to about 1:10. In other embodiments, a single dosage form composition of the invention comprises a weight ratio of proteins to fats that is about 1:0.1 to about 1:9, or about 1:1 to about 1:10. In other embodiments, a single dosage form composition of the invention comprises a weight ratio of proteins to fats that is about 1:0.1 to about 1:7.5, about 1:1 to about 1:7.5, or about 1:2.5 to about 1:10. In other embodiments, a single dosage form composition of the invention comprises a weight ratio of proteins to fats that is about 1:0.1 to about 1:5, about 1:1 to about 1:5, about 1:2.5 to about 1:7.5, or about 1:5 to about 1:10. In other embodiments, a single dosage form composition of the invention comprises a weight ratio of proteins to fats that is about 1:0.1 to about 1:2.5, about 1:1 to about 1:2.5, about 1:2.5 to about 1:5, about 1:5 to about 1:7.5, or about 1:7.5 to about 1:10. In other embodiments, a single dosage form composition of the invention comprises a weight ratio of proteins to fats that is about 1:0.1 to about 1:1. In other embodiments, a single dosage form composition of the invention comprises a weight ratio of proteins to fats that is about 10:0.1 to about 1:0.1.

Various amounts of fats are envisioned. Generally, the amount of fat when present in the single dosage form composition is at least about 0.1 g. For example, a single dosage form composition of the invention may comprise at least about 0.1 g, at least about 0.15 g, at least about 0.2 g, at least about 0.25 g, at least about 0.3 g, at least about 0.35 g, at least about 0.4 g, at least about 0.45 g, or at least about 0.5 g of fats. A single dosage form composition of may also comprise at least about 0.55 g, at least about 0.6 g, at least about 0.65 g, at least about 0.7 g, at least about 0.75 g, at least about 0.8 g, at least about 0.85 g, at least about 0.9 g, at least about 0.95 g of fats. Alternatively, a single dosage form composition of the invention may comprise at least about 1 g, at least about 2.5 g, at least about 5 g, at least about 10 mg, at least about 15 g, at least about 20 g, at least about 25 g, at least about 30 mg, at least about 35 g, at least about 40 g, at least about 45 g, or at least about 50 g of fats. In preferred embodiments, the amount of fats present in a single dosage form composition of the invention is no greater than about 50 g.

In some embodiments, a single dosage form composition of the invention comprises about 1 g to about 50 g of fats. In other embodiments, a single dosage form composition of the invention comprises about 1 g to about 45 g of fats or about 5 g to about 50 g of fats. In other embodiments, a single dosage form composition of the invention comprises about 1 g to about 40 g of fats, about 5 g to about 45 g of fats, or about 10 g to about 50 g. In other embodiments, a single dosage form composition of the invention comprises about 1 g to about 35 g of fats, about 5 g to about 40 g of fats, about 10 g to about 45 g, or about 15 g to about 50 g of fats. In other embodiments, a single dosage form composition of the invention comprises about 1 g to about 30 g of fats, about 5 g to about 35 g of fats, about 10 g to about 40 g, about 15 g to about 45 g of fats, or about 20 g to about 50 g of fats.

In certain embodiments, a single dosage form composition of the invention comprises about 10 g to about 50 g of fats, preferably about 15 g to about 40 g of fats, more preferably about 20 g to about 30 g of fats.

Alternatively, a single dosage form composition of the invention comprises about 10 mg to about 30 mg of fats, preferably about 10 mg to about 25 mg of fats, more preferably about 10 mg to about 20 mg of fats.

In yet another alternative, a single dosage form composition of the invention comprises about 5 g to about 25 g of fats, preferably about 5 g to about 20 g of fats, more preferably about 5 g to about 15 g of fats.

In yet another alternative, a single dosage form composition of the invention comprises about 1 g to about 10 g of fats, preferably about 1 g to about 5 g of fats, more preferably about 2.5 g to about 5 g of fats.

In yet another alternative, a single dosage form composition of the invention comprises about 0.1 g to about 10 g of fats, preferably about 0.1 g to about 5 g of fats, more preferably about 0.1 g to about 1 g of fats.

Methods for determining the amount of fat in a composition are known in the art. A detailed discussion may be found, for example, in “Food Composition Data: Production, Management and Use,” Second edition by Greenfield and Southgate, Food and Agricultural Organization of the United Nations, Rome 2003.

### (i) Additional Components

A single dosage form composition of the invention may further comprise one or more additional components chosen from vitamins, minerals, nutrients, salts, amino acids, plant extracts, flavorings, probiotics, waxes and excipients.

Vitamins, minerals and nutrients may be added individually or in any combination. Non-limiting examples of suitable vitamins, minerals and nutrients include, but are not limited to, fat soluble vitamins, B vitamins, B-complex vitamins and analogs thereof, vitamin A, vitamin C, vitamin D, vitamin K, folic acid, para-aminobenzoic acid, niacin, thiamin, inositol, biotin, boron, calcium, chlorides, chromium, copper, fluorine, iodine, iron (e.g. about 1 mg to about 200 mg, preferably about 1 mg to about 50 mg, more preferably about 1 mg to about 30 mg), manganese, molybdenum, phosphate salts, phosphorus, potassium, selenium, sodium, and vanadium.

The amino acid may be an essential amino acid or a non-essential amino acid, as well as any analog or derivative thereof, individually or in any combination. Non-limiting examples of suitable amino acids include methionine, phenylalanine, threonine, tryptophan, valine, isoleucine, leucine, lysine, glutamine, pyroglutamic acid, taurine, arginine, ornithine, histidine, proline, tyrosine, cysteine, cystine, glycine, asparagine, aspartic acid, citrulline and glutathione; creatine; HMB; AKIC; ketoisocaproate (KIC); OKG; N-acetyl cysteine (NAC); alpha-ketoglutarate (AKG); glycocyamine; NADH; acetyl-L-carnitine; pyroglutamic acid; 4-hydroxyisoleucine; aminolevulinic acid (ALA); melatonin; L-Dopa; theanine; 5-hydroxytryptophan (5-HTP); SAM-e; and DMAE. The amount of amino acids added can range from about 0.1 g to about 50 g, from about 1 g to about 25 g, from about 1 g to about 15 g, from about 1 g to about 10 g, or from about 5 g to about 15 g. In certain embodiments, the amino acids added are one or more branch chain amino acids.

Plant extracts for use in a single dosage form composition of the invention include, but are not limited to, extracts of bilberry, black cohosh, cascara, cat's claw, cayenne, chasteberry, Coleus forskohii, citrus aurantium (bitter orange), cranberry, devil's claw, dong quai, echinacea, ephedra, evening primrose oil, feverfew, flaxseed, garlic, ginger, ginkgo, ginseng, goat weed, goldenseal, gotu kola, grape seed, green tea, guarana, hawthorn, hemp, kava kava, kidney beans, kola nut, licorice, milk thistle, maca, mucuna pruriens, naringin, noni, olive leaf, rhodiola, saw palmetto, shitake mushroom, St. John's wort, Tribulus terrestris, valerian, white willow, yerbe mate, yohimbe, and zhi shi, as well as any analog or derivative thereof, individually or in any combination.

Flavorings may be added to a composition of the invention to make it more desirable to the taste. The concentration of flavorings can be adjusted according to need and taste. Flavorings can be any commercially available flavoring compound used to render dietary and nutritional supplements more palatable. Examples of flavorings include, but are not limited to, beef, lamb, chicken, turkey, fish, mint, peppermint, spearmint, cinnamon, nutmeg, cloves, ginger, wintergreen, vanilla, fruit, fruit concentrates, fruit extracts, fruit essences, peppers, chili pepper, cocoa, chocolate, coffee, caramel, espresso, sarsaparilla, sassafras, salt, wild cherry, ginger, nutmeg, malt, grain flavors, paprika, garlic, and other flavorings well known to those of skill in the art.

Compositions of the invention may comprise one or more probiotics. As used herein, probiotics are food-grade microorganisms (alive, including semi-viable or weakened, and/or non-replicating), that, when consumed, have the potential to confer a beneficial health effect. Suitable probiotics include, but are not limited to, certain non-pathogenic strains of Bacillus species, Bifidobacterium species, Enterococcus species, Lactobacillus species, Lactococcus species, Leuconostoc species, Pediococcus species, Propionibacterium species, Saccharomyces species, Streptococcus species, and combinations thereof. Non-limiting examples of suitable Lactobacillus species include L. acidophilus, L. bulgaricus, L. casei, L. crispatus, L. fermentum, L. gasseri, L. helveticus, L. johnsonii, L. lactis, L paracasei, L. plantarum, L. reuteri, L. rhamnosus, L. salivarius, L. zeae, and combinations thereof. Non-limiting examples of suitable Bifidobacterium species include B. adolescentis, B. angulatum, B. animalis, B. bifidum, B. breve, B. catenulatum, B. gallicum, B. infantis, B. longum, B. pseudocatenulatum, B. thermacidophilum, B. thermophilum, and combinations thereof. Non-limiting examples of suitable Bacillus species include Bacillus coagulans. In an exemplary embodiment, a probiotic may include Bacillus coagulans GBI-30, 6086. Non-limiting examples of suitable Lactococcus species include L. lactis. In an exemplary embodiment, a probiotic may include L. lactis ssp. lactis and/or L. lactis ssp. cremoris. Non-limiting examples of suitable Enterococcus species include E. faecium. Non-limiting examples of suitable Streptococcus species include S. salivarius. In an exemplary embodiment, a probiotic may include S. salivarius ssp. thermophilus. Non-limiting examples of suitable Pediococcus species include P. acidilactici. Non-limiting examples of suitable Leuconostoc species include L. mesenteroides. In an exemplary embodiment, a probiotic may include L. mesenteroides ssp. dextranicum. Non-limiting examples of suitable Propionibacterium species include P. freudenreichii. Non-limiting examples of suitable Saccharomyces species include S. boulardii. Generally, a dosage may contain about 10⁸ to about 10¹¹ colony forming units (CFU). Methods for formulating probiotics are well known in the art.

Compositions may further contain one or more excipients including, but not limited to, buffering agents, effervescing compounds, plant- or animal-derived waxes (e.g. carnauba wax, beeswax, rice bran wax, cetyl palmitate, candelilla wax), gelatin, stabilizers, antioxidants, antimicrobials, colorants, sweeteners, and any combination thereof.

### (j) Beneficial Effect on a Measure of Exercise Performance

A single dosage form composition as provided above, and hereby incorporated into this section, may provide a beneficial effect on a measure of exercise performance when ingested by a subject prior to, during, and/or or following exercise, or upon the notice of symptoms of physiological stress that occur as a result of exercise, as compared to a subject that has not ingested the supplement. In certain embodiments, the beneficial effect is additive. For example, component (a) provides “x” effect and component (b) provides “y” effect, and combined the effect is the sum of “x” and “y”. In certain embodiments, the beneficial effect is synergistic. For example, component (a) provides “x” effect and component (b) provides “y” effect, and combined the effect is greater than the sum of “x” and “y”.

In some embodiments, the beneficial effect on a measure of exercise performance is an increase in maximal oxygen consumption (i.e. VO₂ max) in a subject. VO₂ max refers to the maximum amount of oxygen that an individual can utilize during intense or maximal exercise. Methods for measuring VO₂ max are known in the art. For example, VO₂ max may be determined by a graded exercise test while the subject is wearing a mask that allows direct measurement of the volume and gas concentrations of inspired and expired air. Briefly, the velocity or resistance on a treadmill, cycle ergometer or rowing ergometer in increased at regular interval (e.g. 1 min, 2 min, 3 min, 4 min, 5 min, etc.) until exhaustion (i.e. maximal effort). The amount of the increase is statistically significant, and may be at least 0.5%, at least 1%, at least 1.5%, at least 2%, at least 2.5%, at least 3%, at least 3.5%, at least 4%, at least 4.5%, at least 5%, at least 5.5%, at least 6%, at least 6.5%, at least 7%, at least 7.5%, at least 8%, at least 8.5%, at least 9%, at least 9.5%, at least 10%, at least 15%, at least 20%, at least 30%, at least 40%, or at least 50%.

In some embodiments, the beneficial effect on a measure of exercise performance is an increase in lactate threshold and/or time to onset of blood lactate accumulation in a subject. Lactate threshold refers to the point during exercise of increasing intensity at which lactate begins to accumulate above resting levels. Stated another way, lactate threshold refers to the point where lactate clearance is greater than lactate production. In certain embodiments, lactate threshold may be defined as the point preceding an increase in lactate of >1 mM with increases in intensity. Onset of blood lactate accumulation (OBLA) refers to the point at which blood lactate levels reach 4.0 mM during exercise of increasing intensity. Methods for determining a subject's lactate threshold are known in the art. For example, lactate threshold may be determined by a graded exercise test. Briefly, the velocity or resistance on a treadmill, cycle ergometer or rowing ergometer in increased at regular interval (e.g. 1 min, 2 min, 3 min, 4 min, 5 min, etc.) and lactate measurements are taken at each increment. Lactate concentrations are then plotted against each workload interval to produce a lactate performance curve. A sudden or sharp rise in the curve above base level indicates the lactate threshold. VO_{2 max}, maximum heart rate and other physiological kinetics may be measured during the same test. It may be useful to express lactate threshold in relation to these measurements. For example, lactate threshold may be expressed as a percentage of a subject's VO2 max. For example, if VO₂ max occurs at 24 km/h on a treadmill test and a sharp rise in blood lactate concentration above resting levels is seen at 12 km/h then the lactate threshold is said to be 50% VO₂ max. Methods for measuring lactate levels in blood and muscle samples obtained for a subject are known in the art. Non-invasive methods are also known in the art, e.g. using gas-exchange methods or near-infrared spectroscopy. The amount of the increase is statistically significant, and may be at least 0.5%, at least 1%, at least 1.5%, at least 2%, at least 2.5%, at least 3%, at least 3.5%, at least 4%, at least 4.5%, at least 5%, at least 5.5%, at least 6%, at least 6.5%, at least 7%, at least 7.5%, at least 8%, at least 8.5%, at least 9%, at least 9.5%, at least 10%, at least 15%, at least 20%, at least 30%, at least 40%, or at least 50%.

In some embodiments, the beneficial effect on a measure of exercise performance is an increase in maximal lactate accumulation in a subject. Maximal lactate accumulation refers to the point during exercise of constant intensity at which lactate concentrations are the greatest. Briefly, blood lactate concentrations may be measured at rest (e.g. prior to an exercise test), at regular intervals during a non-graded exercise test (e.g. 1 min, 2 min, 3 min, 4 min, 5 min, 6 min, 7 min, 8 min, 9 min, 10 min, etc.), and optionally during active or passive. During a non-graded exercise test, the velocity or resistance on a treadmill, cycle ergometer or rowing ergometer is held constant for a duration of time (e.g. 5 min, 10 min, 20 min, 30 min, 40 min or more). Passive recovery refers to a period of recovery where the subject's body is completely at rest, while active recovery refers to a period of recovery at an exercise intensity below the subject's lactate threshold. Methods for measuring lactate are described above. Blood lactate is then plotted as a function of time. The amount of the increase is statistically significant, and may be at least 0.5%, at least 1%, at least 1.5%, at least 2%, at least 2.5%, at least 3%, at least 3.5%, at least 4%, at least 4.5%, at least 5%, at least 5.5%, at least 6%, at least 6.5%, at least 7%, at least 7.5%, at least 8%, at least 8.5%, at least 9%, at least 9.5%, at least 10%, at least 15%, at least 20%, at least 30%, at least 40%, or at least 50%.

In other embodiments, the beneficial effect on a measure of exercise performance is an increase in the rate of lactate clearance in the subject during and/or after a period of exercise. During and/or after exercise, lactic acid accumulates in muscle, which then gets released into blood circulation and ultimately cleared. Lactate concentrations in muscle, or more preferably in blood, may be measured by methods known in the art in one or more samples obtained from a subject over a period of time (e.g. 5 minutes, 10 minutes, 15 minutes, 20 minutes, 30 minutes, 35 minutes, 40 minutes, 45 minutes, 50 minutes, 55 minutes, 60 minutes or more) during exercise, active recovery, passive recovery, or a combination thereof. These measurements may be used to produce a lactate recovery curve and/or calculate one or more parameter of lactate recovery by methods known in the art (e.g. the rate constant of lactate clearance, time to peak lactate concentration, peak lactate concentration, etc.). The amount of the increase in the rate of lactate clearance is statistically significant, and may be at least 0.5%, at least 1%, at least 1.5%, at least 2%, at least 2.5%, at least 3%, at least 3.5%, at least 4%, at least 4.5%, at least 5%, at least 5.5%, at least 6%, at least 6.5%, at least 7%, at least 7.5%, at least 8%, at least 8.5%, at least 9%, at least 9.5%, at least 10%, at least 15%, at least 20%, at least 30%, at least 40%, or at least 50%.

In other embodiments, the beneficial effect on a measure of exercise performance is decreased glycogen depletion in a subject. Muscle glycogen concentrations may be measured in a plurality of samples obtained from a subject prior to, during and/or after exercise on one or more successive days of training (e.g. 2, 3, 4 or more days). Methods for measuring muscle glycogen concentrations are known in the art and include, but are not limited to, muscle biopsy, MRI, or ultrasound (e.g. Phys Sportsmed., 2014, pp. 45-52, Vol. 42, No. 3). Indirect measurements may also be used, for example, the correlation of glycogen status with calorimetry measurement. Glycogen depletion is evident if glycogen concentrations for a subject are decreased compared to a similarly timed measurement from a preceding day (e.g. both measurements were made immediately preceding exercise, during exercise, or immediately after exercise). The decrease in glycogen depletion is statistically significant, and may be at least 0.5%, at least 1%, at least 1.5%, at least 2%, at least 2.5%, at least 3%, at least 3.5%, at least 4%, at least 4.5%, at least 5%, at least 5.5%, at least 6%, at least 6.5%, at least 7%, at least 7.5%, at least 8%, at least 8.5%, at least 9%, at least 9.5%, at least 10%, at least 15%, at least 20%, at least 30%, at least 40%, or at least 50%. The decrease in glycogen depletion may also be at least 75%, at least 80%, at least 85%, at least 90%, at least 95%, at least 95%, or at least 99%. In certain embodiments, ingestion of a supplement of the invention prevents glycogen depletion (i.e. there is no significant change in muscle glycogen concentrations).

In other embodiments, the beneficial effect on a measure of exercise performance is an increase in creatine and/or phosphocreatine in the subject during and/or after a period of exercise. Creatine and/or phosphocreatine may be measured in a plurality of blood, muscle or urine samples obtained from the subject prior to and during exercise, by methods known in the art. The amount of the increase is statistically significant, and may be at least 0.5%, at least 1%, at least 1.5%, at least 2%, at least 2.5%, at least 3%, at least 3.5%, at least 4%, at least 4.5%, at least 5%, at least 5.5%, at least 6%, at least 6.5%, at least 7%, at least 7.5%, at least 8%, at least 8.5%, at least 9%, at least 9.5%, at least 10%, at least 15%, at least 20%, at least 30%, at least 40%, or at least 50%.

In other embodiments, the beneficial effect on a measure of exercise performance is an increase in net muscle protein balance (NBAL) in the subject during and/or after a period of exercise. NBAL is the difference between muscle protein synthesis (MPS) and muscle protein breakdown (MPB). Thus an increase in MPS and/or a decrease in MPB are necessary for NBAL to increase, leading to accretion of muscle proteins. In a preferred embodiment, protein synthesis rate is measured after a period of exercise. In certain embodiments, total protein synthesis rate is measured. In other embodiments the protein synthesis rate of one or more proteins is measured (e.g. troponins, tropomyosin, myosin, and the like). Protein synthesis rates may be measured at one or a plurality of time points prior to, during exercise and/or after exercise, by methods known in the art. For example, dynamic measures of muscle protein turnover can be determined in muscle tissue using stable isotope methodologies. Alternatively, or in conjunction with the above labeling method, ingestion of deuterated water (D₂O) to assess cumulative incorporation of deuterium into muscle proteins via deuterium exchange through alanine may also be used. Other methods known in the art are also suitable. The amount of the increase is statistically significant, and may be at least 0.5%, at least 1%, at least 1.5%, at least 2%, at least 2.5%, at least 3%, at least 3.5%, at least 4%, at least 4.5%, at least 5%, at least 5.5%, at least 6%, at least 6.5%, at least 7%, at least 7.5%, at least 8%, at least 8.5%, at least 9%, at least 9.5%, at least 10%, at least 15%, at least 20%, at least 30%, at least 40%, or at least 50%.

In other embodiments, the beneficial effect on a measure of exercise performance is an increase in lean body mass in the subject after a period of exercise. Lean body mass may be calculated using the following formula: lean body mass=body weight−(body weight×body fat %). Lean body mass measurements may be taken before a subject begins ingesting a supplement and after a period of exercise to determine if there has been an increase in lean body mass (e.g. about 1 week, about 2 weeks, about 3 weeks, about 4 weeks, about 5 weeks, about 6 weeks or more). Methods for measuring body fat are known in the art. The amount of the increase is statistically significant, and may be at least 0.5%, at least 1%, at least 1.5%, at least 2%, at least 2.5%, at least 3%, at least 3.5%, at least 4%, at least 4.5%, at least 5%, at least 5.5%, at least 6%, at least 6.5%, at least 7%, at least 7.5%, at least 8%, at least 8.5%, at least 9%, at least 9.5%, at least 10%, at least 15%, at least 20%, at least 30%, at least 40%, or at least 50%.

In other embodiments, the beneficial effect on a measure of exercise performance is a decrease in the amount of zonulin in a fecal sample obtained from the subject during and/or after a period of exercise. Zonulin is a protein of the haptoglobin family released from liver and intestinal epithelial cells, and is described in the art as an important physiological modulator of intercellular tight junctions. Increased zonulin concentrations indicate changes in tight junction competency and increased GI permeability. Zonulin concentrations may be measured in a plurality of fecal samples obtained from a subject prior to, during and/or after a period of exercise, by methods known in the art. For example, commercially available ELISA kits may be used to quantify zonulin concentrations. The amount of the decrease is statistically significant, and may be at least 0.5%, at least 1%, at least 1.5%, at least 2%, at least 2.5%, at least 3%, at least 3.5%, at least 4%, at least 4.5%, at least 5%, at least 5.5%, at least 6%, at least 6.5%, at least 7%, at least 7.5%, at least 8%, at least 8.5%, at least 9%, at least 9.5%, at least 10%, at least 15%, at least 20%, at least 30%, at least 40%, or at least 50%.

In other embodiments, the beneficial effect on a measure of exercise performance is an increase in time to exhaustion or fatigue in the subject during a period of exercise. Time to exhaustion may be measured at one or a plurality of time points during exercise by methods known in the art. Non-limiting examples of suitable tests for exhaustion or fatigue include the electromyographic fatigue threshold test, ventilator threshold, and maximal oxygen concentration (VO_{2PEAK}). The amount of the increase is statistically significant, and may be at least 0.5%, at least 1%, at least 1.5%, at least 2%, at least 2.5%, at least 3%, at least 3.5%, at least 4%, at least 4.5%, at least 5%, at least 5.5%, at least 6%, at least 6.5%, at least 7%, at least 7.5%, at least 8%, at least 8.5%, at least 9%, at least 9.5%, at least 10%, at least 15%, at least 20%, at least 30%, at least 40%, or at least 50%.

In other embodiments, the beneficial effect on a measure of exercise performance is an increase in total work done during a period of exercise by various ergometric methods known in the art.

In other embodiments, the beneficial effect on a measure of exercise performance is a decrease in the time to complete an activity. The activity may be defined as a number of repetitions of one or more exercises (e.g. a distance to travel by running, swimming, cycling, or rowing; or combinations thereof.) The amount of the decrease is statistically significant, and may be at least 0.5%, at least 1%, at least 1.5%, at least 2%, at least 2.5%, at least 3%, at least 3.5%, at least 4%, at least 4.5%, at least 5%, at least 5.5%, at least 6%, at least 6.5%, at least 7%, at least 7.5%, at least 8%, at least 8.5%, at least 9%, at least 9.5%, at least 10%, at least 15%, at least 20%, at least 30%, at least 40%, or at least 50%.

In other embodiments, the beneficial effect on a measure of exercise performance is a decrease in fluid accumulation. It has also been demonstrated in athletes who are given creatine and magnesium that there is an increase in body weight, of which approximately half is attributable to an increase in total body water (TBW). This is due to an osmotic effect of creatine and magnesium and can be reliably measured with body impedance spectroscopy (TBW, intracellular water (ICF) and extracellular water (ECF)). The significance of this is that an increase in intracellular hydration acts as an anabolic proliferative signal and favors protein synthesis. (Brilla L, et al; Metabolism, Vol 52, No 9 (September), 2003: pp 1136-1140). The amount of the increase in TBW or ICF or ECF is statistically significant, and may be at least 1%, at least 2%, at least 3%, at least 4%, at least 5%, at least 7.5%, at least 10%, at least 15%, at least 20%, at least 30%, at least 40%, or at least 50%.

In other embodiments, the beneficial effect on a measure of exercise performance is chosen from the ability to attenuate muscle hypoxia, the ability to reduce markers of muscle stress and damage, the ability optimize muscle oxygenation thereby reducing hypoxic stress, the ability to reduce oxidative stress and muscle tissue disruption (e.g. release of myoglobin and CPK I Ho), or the ability to enhance post-exercise recovery. Spiering B A et al. J Strength Cond Res 2008, pp. 1130-1135, Vol. 22, No. 4; Spiering B A et al. J Strength Cond Res 2007, pp. 259-264, Vol. 21, No. 1; Huang et al, Med Sport Sci, 2012, pp. 135-142, Vol. 59; Brilla et al. Metabolism, 2003, pp. 1136-1140, Vol. 52, No. 9.

### (k) Beneficial Effect on a Gastrointestinal Symptom

A single dosage form composition as provided above, and hereby incorporated into this section, may provide a beneficial effect on one or more gastrointestinal symptom when ingested by a subject prior to, during, and/or or following exercise or upon the notice of the symptom, as compared to a subject that has not ingested the supplement, wherein the symptoms occurs as a result of exercise. In certain embodiments, the beneficial effect is additive. For example, component (a) provides “x” effect and component (b) provides “y” effect, and combined the effect is the sum of “x” and “y”. In certain embodiments, the beneficial effect is synergistic. For example, component (a) provides “x” effect and component (b) provides “y” effect, and combined the effect is greater than the sum of “x” and “y”.

In some embodiments, the beneficial effect on one or more gastrointestinal symptom is a decrease in frequency, severity and/or duration of a gastrointestinal symptom in a subject during and/or after a period of exercise, wherein the gastrointestinal symptom is chosen from cramping, diarrhea, bloating, nausea, bloody stools, and combinations thereof. The period of exercise may be at least about 30 minutes. For example, about 30 minutes, about 1 hour, about 1.5 hours, about 2 hours, about 2.5 hours, about 3 hours, about 3.5 hours, about 4 hours, about 4.5 hours, about 5 hours or more. The amount of the decrease is statistically significant, and may be at least 0.5%, at least 1%, at least 1.5%, at least 2%, at least 2.5%, at least 3%, at least 3.5%, at least 4%, at least 4.5%, at least 5%, at least 5.5%, at least 6%, at least 6.5%, at least 7%, at least 7.5%, at least 8%, at least 8.5%, at least 9%, at least 9.5%, at least 10%, at least 15%, at least 20%, at least 30%, at least 40%, or at least 50%.

### II. Methods of Use

In another aspect, the present invention encompasses a method for reducing intestinal barrier dysfunction in a subject during and/or after a period of exercise, the method comprising orally administering to the subject a single dosage form composition comprising component (a) and component (b). Component (a) is a combination of proteins and carbohydrates in a weight ratio that is at least about 0.5:1. Component (b) is magnesium, zinc, and or choline. Suitable compositions are described in detail in the preceding section. In some embodiments, the subject is a human that regularly exercises (e.g. at least about 30 minutes per day, approximately three times per week). In other embodiments, the subject is a human that is regularly involved in moderate levels of exercise (e.g. about 1 to about 3 hours per day of exercise in one or more workout, at least four times per week). In other embodiments, the subject is a human that is regularly involved in high levels of exercise (e.g. 3 or more hours per day of exercise in one or more workout, at least five times per week).

In another aspect, the present invention encompasses a method for reducing the frequency, severity and/or duration of a gastrointestinal symptom in a subject during and/or after a period of exercise, the method comprising orally administering to the subject a single dosage form composition comprising component (a) and component (b), wherein the gastrointestinal symptom is chosen from cramping, diarrhea, bloating, nausea, bloody stools, and combinations thereof. Component (a) is a combination of proteins and carbohydrates in a weight ratio that is at least about 0.5:1. Component (b) is magnesium, zinc, and/or choline. Suitable compositions are described in detail in the preceding section. In some embodiments, the subject is a human that regularly exercises (e.g. at least about 30 minutes per day, approximately three times per week). In other embodiments, the subject is a human that is regularly involved in moderate levels of exercise (e.g. about 1 to about 3 hours per day of exercise in one or more workout, at least four times per week). In other embodiments, the subject is a human that is regularly involved in high levels of exercise (e.g. 3 or more hours per day of exercise in one or more workout, at least five times per week).

In another aspect, the present invention encompasses a method to produce a beneficial effect on a measure of exercise performance in a subject during and/or after a period of exercise, the method comprising orally administering to the subject a single dosage form composition comprising component (a) and component (b). Component (a) is carbohydrates and proteins in a weight ratio that is at least about 0.5:1. Component (b) is magnesium, zinc, and/or choline. Suitable compositions are described in detail in the preceding section, as are beneficial effects on various measures of exercise performance. Preferably, the beneficial effect is chosen from (a) an increase in maximal oxygen consumption in a subject, (b) an increase in lactate threshold and/or time to onset of blood lactate accumulation in a subject, (c) an increase in the rate of lactate clearance in the subject during and/or after a period of exercise, (d) decreased glycogen depletion in a subject, (e) an increase in creatine and/or phosphocreatine in the subject during a period of exercise, (f) an increase in NBAL or protein synthesis rate in the subject during and/or after a period of exercise, (g) a decrease in the amount of zonulin in a fecal sample obtained from the subject during and/or after a period of exercise, (h) an increase in time to exhaustion or fatigue in the subject during a period of exercise, (i) an increase in total work done during a period of exercise, (j) a decrease in fluid accumulation, and (k) combinations thereof. The amount of the increase or decrease is statistically significant, and may be at least 0.5%, at least 1%, at least 1.5%, at least 2%, at least 2.5%, at least 3%, at least 3.5%, at least 4%, at least 4.5%, at least 5%, at least 5.5%, at least 6%, at least 6.5%, at least 7%, at least 7.5%, at least 8%, at least 8.5%, at least 9%, at least 9.5%, at least 10%, at least 15%, at least 20%, at least 30%, at least 40%, or at least 50%.

In some embodiments, the subject is a human that regularly exercises (e.g. at least about 30 minutes per day, approximately three times per week). In other embodiments, the subject is a human that is regularly involved in moderate levels of exercise (e.g. about 1 to about 3 hours per day of exercise in one or more workout, at least four times per week). In other embodiments, the subject is a human that is regularly involved in high levels of exercise (e.g. 3 or more hours per day of exercise in one or more workout, at least five times per week).

In each of the above embodiments, a composition of the invention may be administered before exercise, during exercise, after exercise, or combinations thereof. “Before exercise” refers to within about 24 hours, about 23 hours, about 22 hours, about 21 hours, about 20 hours, about 19 hours, about 18 hours, about 17 hours, about 16 hours, about 15 hours, about 14 hours, about 13 hours, about 12 hours, about 11 hours, about 10 hours, about 9 hours, about 8 hours, about 7 hours, about 6 hours, about 5 hours, about 4 hours, about 3 hours, about 2 hours, about 1 hours, about 45 minutes, about 30 minutes, about 15 minutes, or less hours before the start of exercise. “After exercise refers to within about 15 minutes, about 30 minutes, about 45 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours after completion of exercise.

In each of the above embodiments, a composition may either be administered daily or administered only on or near the day(s) exercise is performed, for a period of 1, 2, 3, 4, 5, 6, 7, 8, 9, 10 weeks or more. Alternatively, a composition may be administered multiple times per day. For example, multiple administrations can be beneficial if a subject engages in moderate or high levels of exercise more than once per day. The dosage can be ingested in a single serving or divided into various servings and taken at intervals.

In each of the above embodiments, the composition may be a composition chosen from Section I. In various embodiments, the composition is a single dosage form composition comprising (a) carbohydrates and proteins, wherein the weight ratio of proteins to carbohydrates is about 0.5:1 to about 2:1, and the amount of proteins is at least about 10 g, and (b) at least about 400 mg to about 1600 mg of magnesium and/or at least about 1 mg to about 150 mg of zinc. In a preferred embodiment, the composition is a single dosage form composition comprising (a) carbohydrates and proteins, wherein the weight ratio of proteins to carbohydrates is about 0.5:1 to about 2:1, and the amount of proteins is at least about 10 g, and (b) at least about 400 mg to about 1600 mg of magnesium, at least about 1 mg to about 150 mg of zinc, and at least about 10 mg to about 3000 mg of choline. In another preferred embodiment, the composition is a single dosage form composition comprising (a) carbohydrates and proteins, wherein the weight ratio of proteins to carbohydrates is about 0.5:1 to about 2:1, and the amount of proteins is at least about 15 g, and (b) at least about 400 mg to about 1600 mg of magnesium, at least about 1 mg to about 150 mg of zinc, and at least about 10 mg to about 3000 mg of choline. In another preferred embodiment, the composition is a single dosage form composition comprising (a) carbohydrates and proteins, wherein the weight ratio of proteins to carbohydrates is about 0.5:1 to about 2:1, and the amount of proteins is at least about 20 g, and (b) at least about 400 mg to about 1600 mg of magnesium, at least about 1 mg to about 150 mg of zinc, and at least about 10 mg to about 3000 mg of choline. In another preferred embodiment, the composition is a single dosage form composition comprising (a) carbohydrates and proteins, wherein the weight ratio of proteins to carbohydrates is about 0.5:1 to about 2:1, and the amount of proteins is at least about 25 g, and (b) at least about 400 mg to about 1600 mg of magnesium, at least about 1 mg to about 150 mg of zinc, and at least about 10 mg to about 3000 mg of choline. In exemplary embodiments, the composition is a single dosage form composition chosen from Table A, B, or C.

The following examples are included to demonstrate preferred embodiments of the invention. It should be appreciated by those of skill in the art that the techniques disclosed in the examples that follow represent techniques discovered by the inventors to function well in the practice of the invention. Those of skill in the art should, however, in light of the present disclosure, appreciate that changes may be made in the specific embodiments that are disclosed and still obtain a like or similar result without departing from the spirit and scope of the invention. Therefore, all matter set forth or shown in the accompanying drawings is to be interpreted as illustrative and not in a limiting sense.

### EXAMPLES

The following examples illustrate various iterations of the invention.

### Example 1

Various amounts and types of carbohydrates, proteins, fats, carnitine, creatine, iron, magnesium and zinc were combined and formulated as a dry powder to be reconstituted as a liquid. In this example, the single dosage form composition is a scoop of the dry powder, which provides the amounts listed in Table A. Alternative dosage forms can be produced that provide similar amounts of ingredients based on the information provided in the tables (e.g. a powder, a liquid, a gel, etc.).

TABLE A
Single Dosage Form Compositions A-E.

| | Single Dosage Form Compositions | | | | | | | | | |
|---|---|---|---|---|---|---|---|---|---|---|
| | A | | B | | C | | D | | E | |
| Carbo- | 25.4 | g | 25.4 | g | 25.4 | g | 25.4 | g | 25.4 | g |
| hydrate | | | | | | | | | | |
| Protein | 22.5 | g | 22.5 | g | 22.5 | g | 22.5 | g | 22.5 | g |
| Fat | | | | | | | | | 10.4 | g |
| Carnitine | | | | | | | 1 | g | 1 | g |
| Creatine | | | | | 2.25 | g | | | 2.25 | g |
| Fe | | | | | | | | | 29 | mg |
| Mg | 535 | mg | | | 535 | mg | 535 | mg | 535 | mg |
| Zn | | | 15 | mg | 15 | mg | 15 | mg | 15 | mg |

### Example 2

Various amounts and types of carbohydrates, proteins, fats, carnitine, creatine, iron, magnesium and zinc were combined and formulated as a dry powder to be reconstituted as a liquid. In this example, the single dosage form composition is a scoop of the dry powder, which provides the amounts listed in Table B. The amount of branched chain amino acids (BCAA) and other amino acids in the various compositions may be increased or decreased by varying the type of protein or by varying the manufacturing process used to produce the protein isolate. In addition, alternative dosage forms can be produced that provide similar amounts of ingredients based on the information provided in the tables (e.g. a powder, a liquid, a gel, etc.).

TABLE B
Single Dosage Form Compositions F-J.

| | Single Dosage Form Compositions | | | | | | | | | |
|---|---|---|---|---|---|---|---|---|---|---|
| | F | | G | | H | | I | | J | |
| Carbo- | 20 | g | 20 | g | 20 | g | 20 | g | 20 | g |
| hydrate | | | | | | | | | | |
| Protein | 35 | g | 35 | g | 35 | g | 35 | g | 35 | g |
| (BCAA) | (6.5 | g) | (6.5 | g) | (6.5 | g) | (6.5 | g) | (6.5 | g) |
| Fat | | | | | | | | | 3.6 | g |
| Carnitine | | | | | | | 2 | g | 2 | g |
| Creatine | | | | | 2.25 | g | | | 2.25 | g |
| Fe | | | | | | | | | 20 | mg |
| Mg | 400 | mg | | | 400 | mg | 400 | mg | 400 | mg |
| Zn | | | 15 | mg | 15 | mg | 15 | mg | 15 | mg |

### Example 3

Various amounts of pea protein, Ferrochel®, zinc glycinate, DimaCal®, tricalcium phosphate (TCP), brown rice isolate, Creatine MagnaPower®, choline bitartrate, and L-carnitine tartrate were combined and formulated as a dry powder to be reconstituted as a liquid (Table C). In this example, the single dosage form composition is a heaped 50 g scoop of the dry powder, which provides the amounts listed in Table D. The amount of branched chain amino acids (BCAA) and other amino acids in various compositions may be increased or decreased by varying the type of protein or by varying the manufacturing process used to produce the protein isolate. In addition, alternative dosage forms can be produced that provide similar amounts of ingredients based on the information provided in the tables (e.g. a powder, a liquid, a gel, etc.).

TABLE C
Compositions K-L.

| Compositions | | |
|---|---|---|
| | K | L |

| | | | | |
|---|---|---|---|---|
| Pea Protein | 31,200 | mg | 31,200 | mg |
| Ferrochel ® | 75 | mg | 100 | mg |
| Zinc glycinate | 75 | mg | 75 | mg |
| DimaCal ® | 300 | mg | 380 | mg |
| TCP | 300 | mg | 370 | mg |
| Brown Rice Isolate | 14,132 | mg | 15250 | mg |
| Creatine MagnaPower | 5,000 | mg | 5,000 | mg |
| Choline Bitartrate | 1415 | mg | 142 | mg |
| L-Carnitine Tartrate | 2990 | mg | 2990 | mg |
| Total | 55,487 | mg | 55,507 | mg |

TABLE D
Single Dosage Form Compositions K-L.

| Single Dosage Form Compositions | | |
|---|---|---|
| | K | L |

| | | | | |
|---|---|---|---|---|
| Carbohydrate | 13 | g | 14 | g |
| Protein (BCAA) | 25 | g | 25 | g |
| | (5.5 | g) | (5.5 | g) |
| Fat | 0.3 | g | 0.3 | g |
| P | 53 | mg | 65 | mg |
| Mg | 400 | mg | 400.6 | mg |
| Fe | 15 | mg | 20 | mg |
| Zn | 15 | mg | 15 | mg |
| Ca | 200 | mg | 250 | mg |
| Choline | 550 | mg | 55 | mg |
| Carnitine | 2 | g | 2 | g |
| Creatine | 2.25 | g | 2.25 | g |

| | | |
|---|---|---|
| Energy (Kcal) | 163 | 174 |

1-74. (canceled)

75. A single dosage form composition listed in Table A or Table B, or a single dosage form composition of a composition listed in Table C.

76. The composition of claim 75, wherein the protein is one or more protein chosen from egg proteins, plant proteins, milk proteins, animal proteins, as well as isolates, concentrates, agglomerates and hydrolysates thereof.

77. The composition of claim 76, wherein the protein is one or more protein chosen from pea proteins, whey proteins, soy proteins, caseinates, egg proteins, or an isolates, a concentrates, an agglomerates or a hydrolysates thereof.

78. The composition of claim 75, wherein the carbohydrate is chosen from ribose, dextrose, maltose, maltodextrin, fructose, galactose, trehalose, isomaltulose, pyruvates, glucosamine, glucose, sucrose, lactose, pectins, carageenan, acacia, tragacanth, guar, xanthan, arabinogalactan, inulin, konjac flour, orate any combinations thereof.

79. The composition of claim 75, wherein the magnesium is chosen from magnesium salt, magnesium glycinate chelate, magnesium glycinate chelate—buffered, magnesium glycinate chelate taste free, dimagnesium malate, magnesium creatine chelate, magnesium aspartate, magnesium glycyl glutamine chelate, magnesium lysyl glycinate chelate, or any combinations thereof.

80. The composition of claim 75, wherein the zinc is chosen from zinc salts, zinc sulfates, zinc chelates, zinc complexes or zinc admixtures.

81. The composition of claim 75, wherein the fats is chosen from sunflower oil, canola oil, safflower, a short chain triglycerides, a medium chain triglycerides, a long chain triglycerides, or any combinations thereof.

82. The composition of claim 75, wherein the creatine is chosen from creatine monohydrate, creatine phosphate, a metal creatine chelate, metal creatinine complex, metal creatinine admixture, or a combination thereof.

83. The composition of claim 75, wherein the carnitine is chosen from L-carnitine, acetyl-L-carnitine, a metal carnitine chelate, a metal carnitine complex, a metal carnitine admixture or any combination thereof.

84. (canceled)

85. The composition of claim 75, wherein the composition further comprises at least one probiotic.

86. The composition of claim 85, wherein at least one probiotic is a Bacillus species, a Bifidobacterium species, a Enterococcus species, a Lactobacillus species, a Lactococcus species, a Leuconostoc species, a Pediococcus species, a Propionibacterium species, a Saccharomyces species, a Streptococcus species, or any combinations thereof.

87-89. (canceled)

90. A supplement, optionally formulated as a powder, a liquid, a gel, or a food, comprising (a) carbohydrates and proteins in a weight ratio of about 0.5:1 to about 2:1, wherein the amount of proteins is at least about 10 g; and (b) at least about 400 mg to about 1600 mg of magnesium and/or at least about 1 mg to about 150 mg of zinc and/or at least about 10 mg to about 3000 mg of choline; wherein the amounts of component (a) and component (b) present in the supplement produces in a subject that has ingested the supplement a beneficial effect on a measure of exercise performance and/or a beneficial effect on one or more gastrointestinal symptom.

91. (canceled)

92. The supplement of claim 90 or 91, wherein the supplement is a single dosage form composition listed in Table A, Table B, or Table C.

93-97. (canceled)

98. The supplement of claim 90, wherein the beneficial effect on a measure of exercise performance is (a) decreased glycogen depletion in the subject during a period of exercise, (b) an increase in maximal oxygen consumption (i.e. VO₂ max) in a subject during a period of exercise, (c) an increase in time to exhaustion or fatigue in the subject during a period of exercise, or (d) an increase in total work done by the subject during a period of exercise.

99. The supplement of claim 90, wherein the beneficial effect on a measure of exercise performance is an increase in creatine and/or phosphocreatine in the subject during a period of exercise.

100. The supplement of claim 90, wherein the beneficial effect on a measure of exercise performance is an increase in protein synthesis rate in the subject during and/or after a period of exercise.

101. The supplement of claim 90, wherein the beneficial effect on a measure of exercise performance is an increase in lean body mass in the subject after a period of exercise.

102. The supplement of claim 90, wherein the beneficial effect on a measure of exercise performance is a decrease in the amount of zonulin in a fecal sample obtained from the subject during and/or after a period of exercise, or (b) a decrease in fluid accumulation after a period of exercise.

103-105. (canceled)

106. The supplement of claim 90, wherein the beneficial effect on one or more gastrointestinal symptom is a decrease in frequency, severity and/or duration of a gastrointestinal symptom in a subject during and/or after a period of exercise, wherein the gastrointestinal symptom is chosen from cramping, diarrhea, bloating, nausea, bloody stools, and combinations thereof

107-113. (canceled)

114. The supplement of claim 90, wherein the choline is a choline salt, a choline precursor, a choline metabolite.

115. The supplement of claim 90, wherein the choline is choline bitartrate, choline chloride, choline dihydrogen citrate, choline salicylate, choline phosphate, choline bicarbonate, choline magnesium trisalicylate, cytidine diphosphate-choline, phosphatidylcholine, or soy lecithin.
